(19) United States
(12) Patent Application Publication
(10) Pub. No.: US 2024/0261437 A1
(43) Pub. Date: Aug. 8, 2024

(54) MODIFIED AAV CAPSIDS AND USES THEREOF

(71) Applicant: Solid Biosciences Inc., Cambridge, MA (US)

(72) Inventor: Peter Pechan, Newton, MA (US)

(21) Appl. No.: 17/767,554

(22) PCT Filed: Oct. 9, 2020
(86) PCT No.: PCT/US2020/054990

§ 371 Date: Apr. 8, 2022

Related U.S. Application Data
(60) Provisional application No. 63/021,712, filed on May 8, 2020.
(60) Provisional application No. 62/913,223, filed on Oct. 10, 2019.

(51) Int. Cl. A61K 48/00; A61P 21/00; C07K 14/005; C07K 14/47; C12N 15/86

(52) U.S. Cl. CPC A61K 48/0058; A61P 21/00; C07K 14/005; C07K 14/4707; C12N 15/86; C12N 2750/14122; C12N 2750/14143; C12N 2750/14152; C12N 2830/008

(57) ABSTRACT

The invention described herein provide a modified VPI capsid enabling preferential targeted expression of gene of interest (GOI) in muscle tissues, as well as recombinant adeno-associated virus (rAAV) with the GOI packaged with the modified VPI capsids, and uses thereof.

### REFERENCE TO RELATED APPLICATIONS

This application is a U.S. national stage application filed under 35 U.S.C. § 371, based on International Patent Application No. PCT/US2020/054990, filed on Oct. 9, 2020, which claims the benefit of the filing dates of U.S. Provisional Patent Application Nos. 62/913,223, filed on Oct. 10, 2019, and 63/021,712, filed on May 8, 2020. The entire contents of each of the foregoing applications are incorporated herein by reference.

### REFERENCE TO SEQUENCE LISTING

This application is being filed with a Sequence Listing in electronic format. The Sequence Listing file, named 129159_01603_SL, was created on Jan. 24, 2024, and is 39,313 bytes in size. The information in electronic format of the Sequence Listing is incorporated herein by reference in its entirety.

### BACKGROUND OF THE INVENTION

Recombinant adeno-associated viral (rAAV) vectors demonstrate great promise as the leading platform for in vivo gene delivery. A variety of rAAV vectors enable delivery to multiple tissues, including the muscular system, for the treatment of many genetic and other complex diseases. Some natural serotypes as well as engineered rAAV capsids exhibit enhanced widespread biodistribution to muscle, which could reduce the total dose required.

Previously, muscular gene delivery was performed by direct injection of rAAV vectors into muscle. More recently, intravenous (IV) administration of rAAV has been increasingly employed to facilitate distribution to many types of muscle and is now the preferred administration route for several clinical trials.

### SUMMARY OF THE INVENTION

One aspect of the invention provides a modified adeno-associate virus (mAAV) capsid polypeptide comprising a polypeptide selected from the group consisting of SEQ ID NOs: 1-12, wherein the polypeptide is inserted between residues 588 and 589 of wild-type AAV9 VPI capsid, or the two corresponding residues of a non-AAV9 wild-type Clad F (e.g., AAVhu.32), a wild-type Clad A (e.g., AAV1/6), or a wild-type Clad E (e.g., AAV8, AAVrh.10, or AAVrh.74) VP1 capsid. That is, other than the polypeptide of any one of SEQ ID NOs: 1-12 inserted between residues 588 and 589 of the wild-type AAV9 VPI capsid, or the two corresponding residues of a non-AAV9 wild-type Clad F (e.g., AAVhu.32), a wild-type Clad A (e.g., AAV6), or a wild-type Clad E (e.g., AAV8, AAVrh.10, or AAVrh.74) VP1 capsid, there is no other sequence changes in the wild-type Clad F (e.g., AAV9 or AAVhu.32), wild-type Clad A (e.g., AAV6), or wild-type Clad E (e.g., AAV8, AAVrh.10, or AAVrh.74) VP1 capsid.

In other words, the invention provides a modified adeno-associate virus (mAAV) capsid polypeptide comprising a polypeptide inserted between residues 588 and 589 of wild-type AAV9 VPI capsid, or the two corresponding residues of a non-AAV9 wild-type Clad F (e.g., AAVhu.32), a wild-type Clad A (e.g., AAV6), or a wild-type Clad E (e.g., AAV8, AAVrh.10, or AAVrh.74) VP1 capsid, wherein said polypeptide inserted between residues 588 and 589 of wild-type AAV9 VPI capsid, or the two corresponding residues of a non-AAV9 wild-type Clad F (e.g., AAVhu.32), a wild-type Clad A (e.g., AAV6), or a wild-type Clad E (e.g., AAV8, AAVrh.10, or AAVrh.74) VP1 capsid, comprises, consists essentially of, or consists of the amino acid sequence of any one of SEQ ID NOs: 1-12.

In certain embodiments, the mAAV capsid polypeptide comprises the polypeptide of SEQ ID NO: 1, 11, or 12.

That is, a specific aspect of the invention provides a modified adeno-associate virus (mAAV) capsid polypeptide comprising, consisting essentially of, or consisting of the polypeptide of SEQ ID NO: 1.

In a related specific aspect, the invention provides a modified adeno-associate virus (mAAV) capsid polypeptide comprising, consisting essentially of, or consisting of the polypeptide of SEQ ID NO: 11.

In yet another specific aspect, the invention provides a modified adeno-associate virus (mAAV) capsid polypeptide comprising, consisting essentially of, or consisting of the polypeptide of SEQ ID NO: 12.

Another aspect of the invention provides a recombinant adeno-associated virus (rAAV, such as a recombinant AAV6, AAV8, AAVrh.74, or AAV9), comprising any one of the subject mAAV capsid polypeptide described herein.

In certain embodiments, the VP1 capsid of the rAAV consists of or consists essentially of any one of the mAAV capsid polypeptide of the invention.

In Certain Embodiments, the rAAV Comprises a Gene of Interest (GOI) Flanked by a Pair of AAV ITR Sequences.

In certain embodiments, the pair of AAV ITR sequences are AAV2, AAV6, AAV8, AAVrh.74, or AAV9 ITR sequences.

In certain embodiments, the gene of interest (GOI) includes a gene responsible for /defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI encodes a microdystrophin.

In certain embodiments, the microdystrophin is one described in U.S. Pat. Nos. 7,906,111; 7,001,761; 7,510,867; 6,869,777; 8,501,920; 7,892,824; PCT/US2016/013733; or U.S. Pat. No. 10,166,272.

In certain embodiments, the microdystrophin comprises a coding sequence for R16 and R17 spectrin-like repeats for the full length dystrophin protein (such as one described in U.S. Pat. No. 7,892,824).

In certain embodiments, the microdystrophin comprises a coding sequence for the R1, R16, R17, R23, and R24 spectrin-like repeats of the full-length dystrophin protein; or a microdystrophin gene described in PCT/US2016/013733 or U.S. Pat. No. 10,479,821.

In certain embodiments, the GOI is operatively linked to a transcriptional regulatory cassette, such as a muscle specific promoter (e.g., a CK8 promoter or a cardiac troponin T (cTnT) promoter).

In certain embodiments, the GOI is a micro-dystrophin gene encoding a protein comprising, from N- to C-terminus, an amino-terminal actin-binding (AB1) domain, a β-dystroglycan binding domain, a Hinge 1 domain (H1), a spectrin-like repeat domain consisting of five spectrin-like repeats that include spectrin-like repeat 1 (SR1), spectrin-like repeat 16 (SR16), spectrin-like repeat 17 (SR17), spectrin-like repeat 23 (SR23), and spectrin-like repeat 24 (SR24), and a Hinge 4 domain (H4), wherein the micro-dystrophin gene is operatively linked to a muscle-specific human muscle creatine kinase CK8 promoter (e.g., SEQ ID NO:19 of U.S. Pat. No. 10,479,821), and wherein the GOI is flanked by a pair of AAV2 ITR (inverted terminal repeat) sequence.

Another aspect of the invention provides a polynucleotide encoding the modified adeno-associate virus (mAAV) capsid polypeptide of the invention, or a polypeptide sequence at least 95%, 96%, 97%, 98%, or 99% identical thereto.

In certain embodiments, the polynucleotide is codon-optimized for mammalian expression.

Another aspect of the invention provides a vector comprising the polynucleotide of the invention.

In certain embodiments, the vector is a plasmid or a viral vector (such as an HSV vector, or an AAV vector).

Another aspect of the invention provides a cultured host cell comprising: (a) a recombinant nucleic acid molecule encoding: (1) the modified adeno-associate virus (mAAV) capsid polypeptide of the invention, or (2) a sequence at least 95%, 96%, 97%, 98%, or 99% identical to (1), wherein the recombinant nucleic acid molecule optionally further comprises a heterologous non-AAV sequence; or, (b) a recombinant adeno-associated virus (rAAV) of the invention.

Another aspect of the invention provides a method of treating a disease or condition such as muscular dystrophy in a subject in need thereof, the method comprises administering to the subject a therapeutically effective amount of any one of the rAAV of the invention.

In certain embodiments, when compared to an otherwise identical reference rAAV with wild-type AAV9 VPI capsid, the GOI of the rAAV is preferentially expressed in cardiac muscle, skeletal muscle, and/or smooth muscle (e.g., smooth muscle in diaphragm).

In certain embodiments, when compared to an otherwise identical reference rAAV with wild-type AAV9 VPI capsid, the GOI of the rAAV is expressed in liver at a statistically significantly lower level.

In certain embodiments, the muscular dystrophy is DMD (Duchenne Muscular Dystrophy) or BMD (Becker Muscular Dystrophy).

In certain embodiments, (1) the muscular dystrophy is LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or spinal muscular atrophy or SMA; (2) the disease or condition is Sanfilippo syndrome or mucopolysaccharidosis type IIIB (MPS IIIB), mucopolysaccharidosis type IIIA or MPS IIIA, Pompe disease, or Fabry disease; or, (3) the disease or condition is characterized or caused by a genetic defect in a gene encoding Factor IX, Factor VIII, Myotubularin 1 (MTM1), GalNAc transferase GALGT2, calpain-3 (CAPN-3), or glucocerebrosidase.

Another aspect of the invention provides a method of producing rAAV, wherein the rAAV comprises any one of the mAAV capsid polypeptide of the invention, the method comprising introducing a rAAV vector encoding a GOI flanked by a pair of ITR sequences to a producing or packaging cell line expressing any one of the mAAV capsid polypeptide of the invention.

In certain embodiments, the producing or packaging cell line is infected by an HSV vector encoding the mAAV capsid polypeptide of the invention.

In certain embodiments, the producing or packaging cell line is transfected by endoing sequence encoding the mAAV capsid polypeptide of the invention.

In certain embodiments, the producing or packaging cell line is HEK293 cells, A549 cells, or HeLa cells.

It should be understood that any one embodiment of the invention, including those described only in the examples or under one aspect of the invention, can be combined with any one or more other embodiment of the invention unless explicitly disclaimed or improper.

### BRIEF DESCRIPTION OF THE DRAWINGS

FIGS. 1A-1E show in vitro potency assay comparing AAV9 and AAV8 with the subject AAV-SLB101 capsid (abbreviated in the figures as “AAV-101” or referred to as “SLB101,” “SLB-101” or variations thereof elsewhere).

FIGS. 2A and 2B show in vitro potency assay comparing AAV9 and AAV8 with the subject AAV-SLB101 capsid, on both C2C12 cells and patient-derived DMD Mouly cells.

FIGS. 3A-3C show favorable biodistribution and higher level of microdystrophin expression in all three muscle tissues vs. liver tissue, with the subject rAAV capsid as compared to AAV9, and the lack of difference in expression in peripheral tissues such as lung, spleen, kidney, and brain.

FIG. 4 shows production yield of the novel capsids of the invention compared to wild-type AAV9 capsid. Expanded panel of the subject AAV capsids (AAV-SLB101 to AAV-SLB112) were compared to wild-type AAV9 for AAV yield from triple transfection of adherent 293T cells, and purification via step-wise iodixanol ultracentrifugation. Two additional modified capsids AAV-SLB113 and -SLB114 (see sequence alignments in FIG. 6 below) were also included in the comparison.

FIGS. 5A-5C show results of in vitro characterization of the subject modified AAV capsids for comparison to wild-type AAV9 capsid in C2C12 Cells. Specifically, FIG. 5A shows quantification of AAV capsid binding to the cell surface of C2C12 cells as measured by qPCR of DNA isolated after 1 hour of incubation at 4° C. AAV-SLB101 (p<0.0001), -112 (p<0.01) and -114 (p<0.001) bind to C2C12 cells significantly more than AAV9. Statistics are determined by ordinary one-way ANOVA. FIG. 5B shows quantification of uptake of AAV into C2C12 cells as measured by qPCR of DNA isolated after 1 hour of incubation at 4° C. followed by an additional hour at 37° C. AAV-SLB101, 102, 108, 111, 112, 113, 114 were taken up by C2C12 cells significantly more than AAV9 (p<0.0001). Statistics are determined by ordinary one-way ANOVA. In FIG. 5C, C2C12 cells were transduced with microdystrophin-expressing vectors packaged in AAV9, AAV-SLB101 and thirteen additional capsids (AAV-SLB 102 to AAV-SLB114). Cells were harvested 96 hours after transduction and microdystrophin expression was measured. The data shown are normalized to AAV9 and fold change is indicated in the table. AAV-SLB101, 102, 111, 112 and 113 had the highest microdystrophin protein expression over AAV9 (p<0.0001), with AAV-SLB109 and 114 resulting in only slightly higher expression than AAV9 (p<0.001). Statistics are determined by ordinary one-way ANOVA.

FIG. 6 shows sequence alignment of wild-type AAV9 and SLB101-SLB 114 (the 2nd to the 16th line, respectively) and the consensus sequence (top 1st line sequence). A smaller regional alignment among wild-type AAV9, SLB101, SLB-113, and SLB114 is also included.

FIG. 7 shows biodistribution of AAV9 variant viruses with the variant capsids SLB-101, -102, -113, and -114 as compared to wild-type AAV9, in the heart, quad, and diaphragm muscles, as well as in the liver. Different levels of statistical significance are indicated with “*”

FIG. 8 shows extended biodistribution of AAV9 variant viruses with the variant capsids SLB-101, -102, -113, and -114 as compared to wild-type AAV9, in the lung, kidney, brain, and spleen. Different levels of statistical significance are indicated with “*”

FIG. 9 shows expression of microdystrophin encoded by AAV9 vectors with the variant AAV9 capsids SLB-101, -102, -113, and -114, as compared to wild-type AAV9. Different levels of statistical significance are indicated with “*”

### DETAILED DESCRIPTION OF THE INVENTION

### 1. Overview

The invention described herein provides an optimized delivery of gene of interest (GOI), partly based on an optimized rAAV capsid that exhibits superior biodistribution than wild-type capsids. Such rAAV having modified VPI capsids can be used to preferentially deliver GOI to muscle tissues at higher expression level and/or lower dose, thus facilitating more successful gene therapy targeting muscle tissues (e.g., skeletal, cardiac, and/or smooth muscle tissues).

In particular, according to one aspect of the invention, provided herein is modified AAV capsids and methods for AAV capsid selection that enable preferential targeted delivery of GOI to muscle tissues and muscle cell types.

According to another aspect of the invention, provided herein is a method for selecting a potent infectious AAV capsid for potential lowering of an effective AAV dose required for successful gene therapy.

As rAAV drug products advance clinically, analytical tools to characterize these complex products are needed. Examples include detection and quantification of mammalian cell host or viral helper DNA by PCR, qPCR, denaturing gel electrophoresis, and Southern blot. Development and validation of these and additional assays will enable full characterization of rAAV vectors. Thus yet another aspect of the invention provides an analytical tool for characterizing the subject rAAV capsids, vectors, and products, such as the ones described in the Examples.

A further aspect of the invention provides an in vitro potency assay as a critical tool for the development of the subject rAAV capsids and vectors. rAAV vectors with capsids of various natural serotypes and engineered capsids are compared to estimate their efficacy for systemic administration, and their potential for the development of treatment for disease indications treatable by gene therapy, such as Duchenne muscular dystrophy (DMD). In applications that require systemic administration of high doses of vectors, such as Duchenne muscular dystrophy, these analytical tools and assays have the potential to further characterize and improve rAAV products, and thereby improve safety and efficacy of gene transfer therapy.

The detailed aspects of the invention are described further below.

### 2. Modified VP1 Capsids

The modified AAV9 VP1 capsid protein of the invention has a short peptide of about 7-13 residues inserted between residues Q588 and A589 of the wild-type AAV9 VP1 capsid protein. In certain embodiments, the short peptide is any one of SEQ ID NOs: 1-12.

(SEQ ID NO: 1)
RGDLGLS
(also known as SLB101)
(SEQ ID NO: 2)
RGDMSRE
(also known as SLB102)
(SEQ ID NO: 3)
GEARISA
(also known as SLB103)
(SEQ ID NO: 4)
ESGLSQS
(also known as SLB104)
(SEQ ID NO: 5)
EYRDSSG
(also known as SLB105)
(SEQ ID NO: 6)
DLGSARA
(also known as SLB106)
(SEQ ID NO: 7)
SGNSGAA
(also known as SLB107)
(SEQ ID NO: 8)
CDCRGDCFC
(also known as SLB108)
(SEQ ID NO: 9)
NDVRSAN
(also known as SLB109)
(SEQ ID NO: 10)
NDVRAVS
(also known as SLB110)
(SEQ ID NO: 11)
GGGRGDLGLSGGG
(also known as SLB111)
(SEQ ID NO: 12)
GGSRGDLGLSGGS
(also known as SLB112)

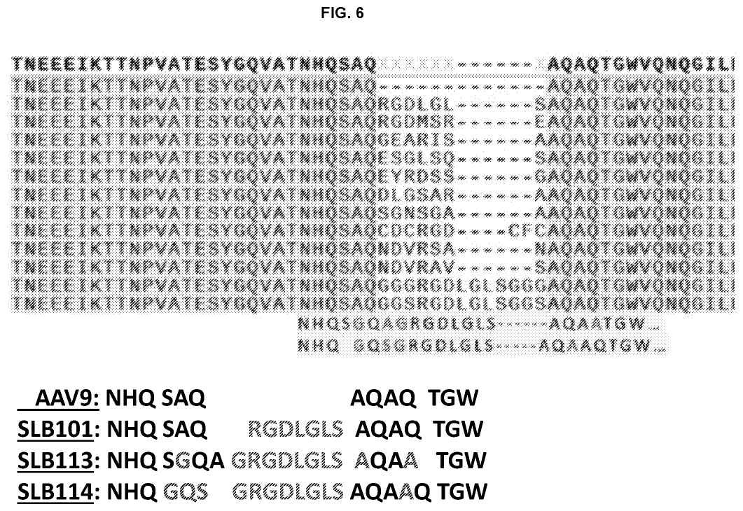

Two additional sequences with similar insertion modifications but different surrounding/flanking sequences are referred to as SLB113 and SLB114 (see FIG. 6).

Wile-type AAV9 VP1 capsid protein sequence is known in the art, and is copied below. The residues Q588 and A589, between which one of SEQ ID NO: 1-12 is inserted, are double underlined.

(SEQ ID NO: 13)
MAADGYLPDWLEDNLSEGIREWWALKPGAPQPKANQQHQDNARGLVLPG
YKYLGPGNGLDKGEPVNAADAAALEHDKAYDQQLKAGDNPYLKYNHADA
EFQERLKEDTSFGGNLGRAVFQAKKRLLEPLGLVEEAAKTAPGKKRPVE
QSPQEPDSSAGIGKSGAQPAKKRLNFGQTGDTESVPDPQPIGEPPAAPS
GVGSLTMASGGGAPVADNNEGADGVGSSSGNWHCDSQWLGDRVITTSTR
TWALPTYNNHLYKQISNSTSGGSSNDNAYFGYSTPWGYFDFNRFHCHFS
PRDWQRLINNNWGFRPKRLNFKLFNIQVKEVTDNNGVKTIANNLTSTVQ
VFTDSDYQLPYVLGSAHEGCLPPFPADVEMIPQYGYLTLNDGSQAVGRS
SFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVS
TTVTQNNNSEFAWPGASSWALNGRNSLMNPGPAMASHKEGEDRFFPLSG
SLIFGKQGTGRDNVDADKVMITNEEEIKTTNPVATESYGQVATNHQSA [symbol]
QAQTGWVQNQGILPGMVWQDRDVYLQGPIWAKIPHTDGNFHPSPL
MGGFGMKHPPPQILIKNTPVPADPPTAFNKDKLNSFITQYSTGQVSVE
IEWELQKENSKRWNPEIQYTSNYYKSNNVEFAVNTEGVYSEPRPIGTRY
LTRNL

In certain embodiments, the modified AAV9 VP1 capsids of the invention has the polypeptide of SEQ ID NO: 1 (bold italic) inserted between residues Q588 and A589 (both double underlined) of SEQ ID NO: 13 as following:

(SEQ ID NO: 14)
MAADGYLPDWLEDNLSEGIREWWALKPGAPQPKANQQHQDNARGLVLPGYKYLGPGN
GLDKGEPVNAADAAALEHDKAYDQQLKAGDNPYLKYNHADAEFQERLKEDTSFGGNLGRAVE
QAKKRLLEPLGLVEEAAKTAPGKKRPVEQSPQEPDSSAGIGKSGAQPAKKRLNFGQTGDTES
VPDPQPIGEPPAAPSGVGSLTMASGGGAPVADNNEGADGVGSSSGNWHCDSQWLGDRVITTS
TRTWALPTYNNHLYKQISNSTSGGSSNDNAYFGYSTPWGYFDFNRFHCHESPRDWQRLINNN
WGFRPKRLNFKLFNIQVKEVTDNNGVKTIANNLTSTVQVFTDSDYQLPYVLGSAHEGCLPPF
PADVEMIPQYGYLTLNDGSQAVGRSSFYCLEYFPSQMLRTGNNFQFSYEFENVPFHSSYAHS
QSLDRLMNPLIDQYLYYLSKTINGSGQNQQTLKFSVAGPSNMAVQGRNYIPGPSYRQQRVST
TVTQNNNSEFAWPGASSWALNGRNSLMNPGPAMASHKEGEDRFFPLSGSLIFGKQGTGRDNV
DADKVMITNEEEIKTTNPVATESYGQVATNHQSA [symbol] QAQTGWVQNQGILPGMVWQ
DRDVYLQGPIWAKIPHTDGNFHPSPLMGGFGMKHPPPQILIKNTPVPADPPTAFNKDKLNSF
ITQYSTGQVSVEIEWELQKENSKRWNPEIQYTSNYYKSNNVEFAVNTEGVYSEPRPIGTRYL
TRNL

In certain embodiments, the modified AAV9 VP1 capsids of the invention has the polypeptide of SEQ ID NO: 11 (bold italic) inserted between residues Q588 and A589 (both double underlined) of SEQ ID NO: 13 as following:

(SEQ ID NO: 15)
MAADGYLPDWLEDNLSEGIREWWALKPGAPQPKANQQHQDNARGLVLPGYKYLGPGN
GLDKGEPVNAADAAALEHDKAYDQQLKAGDNPYLKYNHADAEFQERLKEDTSFGGNLGRAVE
QAKKRLLEPLGLVEEAAKTAPGKKRPVEQSPQEPDSSAGIGKSGAQPAKKRLNFGQTGDTES
VPDPQPIGEPPAAPSGVGSLTMASGGGAPVADNNEGADGVGSSSGNWHCDSQWLGDRVITTS
TRTWALPTYNNHLYKQISNSTSGGSSNDNAYFGYSTPWGYFDFNRFHCHFSPRDWQRLINNN
WGFRPKRLNFKLFNIQVKEVTDNNGVKTIANNLTSTVQVFTDSDYQLPYVLGSAHEGCLPPF
PADVFMIPQYGYLTLNDGSQAVGRSSFYCLEYFPSQMLRTGNNFQFSYEFENVPFHSSYAHS
QSLDRLMNPLIDQYLYYLSKTINGSGQNQQTLKFSVAGPSNMAVQGRNYIPGPSYRQQRVST
TVTQNNNSEFAWPGASSWALNGRNSLMNPGPAMASHKEGEDRFFPLSGSLIFGKQGTGRDNV
DADKVMITNEEEIKTTNPVATESYGQVATNHQSA [symbol] QAQTGWVQNQGIL
PGMVWQDRDVYLQGPIWAKIPHTDGNFHPSPLMGGFGMKHPPPQILIKNTPVPADPPTAENK
DKLNSFITQYSTGQVSVEIEWELQKENSKRWNPEIQYTSNYYKSNNVEFAVNTEGVYSEPRP
IGTRYLTRNL

In certain embodiments, the modified AAV9 VP1 capsids of the invention has the polypeptide of SEQ ID NO: 12 (bold italic) inserted between residues Q588 and A589 (both double underlined) of SEQ ID NO: 13 as following:

(SEQ ID NO: 16)
MAADGYLPDWLEDNLSEGIREWWALKPGAPQPKANQQHQDNARGLVLPGYKYLGPGN
GLDKGEPVNAADAAALEHDKAYDQQLKAGDNPYLKYNHADAEFQERLKEDTSFGGNLGRAVE
QAKKRLLEPLGLVEEAAKTAPGKKRPVEQSPQEPDSSAGIGKSGAQPAKKRLNFGQTGDTES
VPDPQPIGEPPAAPSGVGSLTMASGGGAPVADNNEGADGVGSSSGNWHCDSQWLGDRVITTS
TRTWALPTYNNHLYKQISNSTSGGSSNDNAYFGYSTPWGYFDENRFHCHESPRDWQRLINNN
WGFRPKRLNFKLFNIQVKEVTDNNGVKTIANNLTSTVQVFTDSDYQLPYVLGSAHEGCLPPF
PADVFMIPQYGYLTLNDGSQAVGRSSFYCLEYFPSQMLRTGNNFQFSYEFENVPFHSSYAHS
QSLDRLMNPLIDQYLYYLSKTINGSGQNQQTLKFSVAGPSNMAVQGRNYIPGPSYRQQRVST
TVTQNNNSEFAWPGASSWALNGRNSLMNPGPAMASHKEGEDRFFPLSGSLIFGKQGTGRDNV
DADKVMITNEEEIKTTNPVATESYGQVATNHQSA [symbol] QAQTGWVQNQGIL
PGMVWQDRDVYLQGPIWAKIPHTDGNFHPSPLMGGFGMKHPPPQILIKNTPVPADPPTAFNK
DKLNSFITQYSTGQVSVEIEWELQKENSKRWNPEIQYTSNYYKSNNVEFAVNTEGVYSEPRP
IGTRYLTRNL

The modified AAV9 VP1 capsids of the invention enable preferential targeted expression of GOI from rAAV packaged in such modified AAV9 VP1 capsids, especially in muscle tissues, including cardiac muscles (i.e., muscles in the heart), skeletal muscles (e.g., the quadriceps), and/or smooth muscles (e.g., the diaphragm muscles).

While not wishing to be bound by any particular theory, changing the wild-type amino acid sequences in the AAV9 VP1 capsid sequence around the insertion point (i.e., 1, 2, 3, 4, 5, 6, 7, or 8 residues N-terminal to Q588 and/or Q588 itself, and/or 1, 2, 3, 4, 5, 6, 7, or 8 residues C-terminal to A589 and/or A589 itself) may affect (e.g., adversely affect) tissue targeting and/or expression level of the GOI in the target tissue. One embodiment of this affect or change may be the tissue infection rate or expression level in a desirable and particular tissue(s), e.g., any of the muscle tissues such as cardiac, skeletal, and/or smooth muscle tissue(s), when compared with the AAV packaged with the subject modified AAV9 VP1 capsids that have completely preserved the wild-type AAV9 VPI capsid sequences around and including residues Q588 and A589.

In certain embodiments, the preferential expression of the GOI, compared to an otherwise identical rAAV construct except for using wild-type AAV9 capsids, is at least 50% higher, 100% higher (i.e., 2-fold), 3-fold, 5-fold, 10-fold, 12-fold, 15-fold, 20-fold, 22-fold, 25-fold, 30-fold, 50-fold, 75-fold, or 100-fold or more. In certain embodiments, the fold increase is measured based on an in vitro assay and/or an in vivo assay substantially as those described in the Examples (incorporated here by reference for clarity and to avoid redundancy). For example, in certain embodiments, the in vitro cell line is C2C12 or patient-derived Mouly cells. In certain embodiments, the in vivo assay is conducted in the mdx mouse model of DMD.

In certain embodiments, the modified adeno-associate virus (mAAV) capsid polypeptide is based not on AAV9, but on wild-type sequence of a non-AAV9 wild-type Clad F (e.g., AAVhu.32), a wild-type Clad A (e.g., AAV6), or a wild-type Clad E (e.g., AAV8, AAVrh.10, or AAVrh.74) VP1 capsid. That is, the polypeptide selected from the group consisting of SEQ ID NOs: 1-12 is inserted between the two corresponding residues of a non-AAV9 wild-type Clad F (e.g., AAVhu.32), a wild-type Clad A (e.g., AAV6), or a wild-type Clad E (e.g., AAV8, AAVrh.10, or AAVrh.74) VP1 capsid.

As used herein, “corresponding residues” refers to the residues in a non-AAV9 wild-type Clad FAAV (e.g., AAVhu.32), a wild-type Clad A AAV (e.g., AAV6), or a wild-type Clad E AAV (e.g., AAV8, AAVrh.10, AAVrh.37, or AAVrh.74) VP1 capsid that aligns with wild-type AAV9 VPI capsid residues 588 and 589, respectively.

As used herein, a non-AAV9 wild-type Clad F AAV includes AAVhu.32. A wild-type Clad A AAV includes AAV1/6, AAVhu.48R2, AAVhu.48R3, and AA Vhu.44R3. A wild-type Clad E AAV includes AAV8, AAVhu.37, AAVrh. 10, AAVpi.2, AAVrh.64R1, AAVrh.64R2, AAVrh.2R, AAVrh.74, and AAVrh.43.

For example, the insertion points in the representative Clad A AAV6, Clad E AAV8 and AAVrh.74, and Clad F AAV9 and AAVhu.32 are shown below. Note that due to the sequence difference, AAV8 residues N590 and T591 “corresponds to the AAV9 residues Q588 and A589, respectively.

| | |
|---|---|
| AAV9 (Clad F) | NHQSAQ⁵⁸⁸_⁵⁸⁹AQAQTGW |
| AAV8 (Clad E) | NLQQQN⁵⁹⁰_⁵⁹¹TAPQIGT |
| AAVrh.10 (Clad E) | NLQQQN⁵⁹⁰_⁵⁹¹AAPIVGA |
| AAVrh.74 (Clad E) | NLQQQN⁵⁹⁰_⁵⁹¹AAPIVGA |
| AAVrh.37 (Clad E) | NLQQQN⁵⁹⁰_⁵⁹¹TGPIVGN |
| AAV6 (Clad A) | NLQSSS⁵⁸⁸_⁵⁸⁹TDPATGD |

3. Gene of Interest (GOI) in rAAV and Treatable Diseases

The recombinant AAV vectors of the invention having the modified VPI capsids may carry any gene of interest (GOI) for treating a disease or condition through gene therapy. The GOI can be any gene or coding sequence within the packaging capacity of the rAAV, e.g., about 4-5 kb, or about 4.7 kb including the ITR sequences, or about 4.4 kb without accounting for the ITR sequences.

In certain embodiments, the rAAV carrying the GOI can be used in gene therapy to treat a disease or condition caused by lacking of function of an endogenous gene in the host, such as a defective version of the GOI.

As used herein, “gene of interest” or GOI generally refers to a nucleic acid or polynucleotide sequence, such as a gene, an open reading frame (ORF), or a coding sequence for protein or RNA such as siRNA, miRNA, shRNA, etc. However, in certain circumstances or context, the term GOI also loosely refers to a protein (encoded by the GOI), or a disease or indication that can be remedied by the GOI, or a disease or indication can be (but is not necessarily) caused by loss of function of the GOI.

For example, the gene GALGT2 encodes the protein GalNAc transferase (β-1,4-N-acetylgalactosamine galactosyltransferase), which is an enzyme that transfers a complex sugar molecule onto a few specific proteins, including dystroglycan. Under normal circumstances, GalNAc transferase is found only at the neuromuscular junction (NMJ), where some components of the dystroglycan-associated protein complex are different than elsewhere in muscle. Importantly, at the NMJ, utrophin is present instead of dystrophin. In the mdx mouse model of muscular dystrophy, viral gene transfer of GALGT2 results in expression of GalNAc transferase across the entire muscle membrane, instead of just at the normal expression domain of the NMJ, as well as upregulation of utrophin across the entire muscle fiber. In the mdx mouse, this expression can correct muscle functional deficits to the same degree as does microdystrophin gene expression. Furthermore, overexpression of GALGT2 corrects muscle pathology in mouse models of other muscular dystrophies, including LGMD2A and congenital muscular dystrophy (MDCIA). Thus GALGT2 is a GOI for treating muscular dystrophy such as DMD, BMD, LGMD2A and MDCIA, even though GALGT2 is not necessarily defective per se in the patient in need of treatment.

In another example, Sarcolipin (SLN) inhibits the sarco/endoplasmic reticulum (SR) Ca²⁺ ATPase (SERCA), and is abnormally elevated in the muscle of DMD patients and animal models such as the mdx mouse model of DMD. Reducing SLN levels by AAV9-mediated RNA interference ameliorates dystrophic pathology in the severe dystrophin/utrophin double mutant (mdx:utr) mouse model of DMD, including attenuation of muscle pathology and improvement of diaphragm, skeletal muscle and cardiac function. Thus the coding sequence for SLN RNAi is a GOI that remedies DMD.

Thus the GOI can be a gene (or protein) that, when expressed, replaces a mutated, damaged, or inactive gene or protein. The GOI can be a gene (or protein) that, when expressed, assists an already functioning process that requires modification for therapy in a disease, disorder, or dysfunction. The GOI can be a gene (or protein) that, when expressed, assists a dysfunctional process that requires modification for therapy in a disease, disorder, or dysfunction. A GOI nucleic acid sequence can be DNA, RNA, or synthetic nucleic acid molecule. The GOI can be a protein, an enzyme, a structural protein, a functional protein, or an adaptable protein based on cell function(s). The GOI can provide therapeutic benefit or a treatment modality for a disease, disorder, or dysfunction.

In certain embodiments, the GOI may be CRISPR-Cas9, Cas 13, TALEN, or other genetic based gene editing protein that are required for intracellular delivery for their intended activity.

Any and all GOIs as used herein may require codon optimization for enhanced expression and activity via known computer based algorithms.

Thus the rAAV that may be produced by using the subject viral capsids (e.g., modified VPI capsids) and may encode a gene of interest (GOI) useful for, e.g., gene therapy to treat a disease or condition. Representative (non-limiting) gene of interest (GOI) may include: a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a microdystrophin gene.

In certain embodiments, the microdystrophin gene is any one described in the following patents: U.S. Pat. Nos. 7,906,111; 7,001,761; 7,510,867; 6,869,777; 8,501,920; 7,892,824; PCT/US2016/013733; U.S. Pat. No. 10,166,272 (all incorporated herein by reference). In certain embodiments, the microdystrophin gene is capable of being packaged into a rAAV virion, e.g., no more than about 4.7 kb in size.

In certain embodiments, the microdystrophin gene contains within its coding sequence spectrin-like repeats R16 and R17 that are capable of restoring nitric oxide synthase (nNOS) activity to the sarcolemma (such as those described in U.S. Pat. No. 7,892,824).

In certain embodiments, the microdystrophin gene comprises a coding sequence for the R1, R16, R17, R23, and R24 spectrin-like repeats (i.e., SR1, SR16, SR17, SR23, and SR24, respectively) of the full-length dystrophin protein, such as one described in PCT/US2016/013733 (incorporated herein by reference). In certain embodiments, the microdystrophin gene does not encode any other spectrin repeats of the full-length dystrophin protein, other than SR1, SR16, SR17, SR23, and SR24.

In certain embodiments, the microdystrophin gene is described in U.S. Pat. Nos. 7,906,111; 7,001,761; 7,510,867; 6,869,777; 8,501,920; 7,892,824; or U.S. Pat. No. 10,166,272, or in PCT/US2016/013733 (all incorporated herein by reference). For example, PCT/US2016/013733 (WO2016/115543A2) provides a micro-dystrophin gene operatively connected to a regulatory cassette, wherein the micro-dystrophin gene encodes a protein comprising: an amino-terminal actin-binding domain; a β-dystroglycan binding domain; and a spectrin-like repeat domain, comprising at least four spectrin-like repeats, wherein two of the at least four spectrin-like repeats comprise a neuronal nitric oxide synthase binding domain. In certain embodiments, the at least four spectrin-like repeats include spectrin-like repeat 1 (SR1), spectrin-like repeat 16 (SR16), spectrin-like repeat 17 (SR17), and spectrin-like repeat 24 (SR24). In certain embodiments, the protein encoded by the micro-dystrophin gene further comprises at least a portion of a hinge domain, such as at least one of a Hinge 1 domain, a Hinge 2 domain, a Hinge 3 domain, a Hinge 4 domain, and a hinge-like domain. In certain embodiments, the micro-dystrophin gene comprises, in N- to C-terminal order: a Hinge 1 domain (H1); a spectrin-like repeat 1 (SR1); a spectrin-like repeat 16 (SR16); a spectrin-like repeat 17 (SR17); a spectrin-like repeat 24 (SR24); and a Hinge 4 domain (H4). In certain embodiments, H1 is directly coupled to the SR1. In certain embodiments, SR 1 is directly coupled to SR16. In certain embodiments, SR16 is directly coupled to SR17. In certain embodiments, SR 17 is directly coupled to SR24. In certain embodiments, SR24 is directly coupled to the H4. In certain embodiments, the protein encoded by the micro-dystrophin gene further comprises between SR1 and SR16, in N- to C-terminal order, a spectrin-like repeat 2 (SR2) and a spectrin-like repeat 3 (SR3). In certain embodiments, SR1 is directly coupled to SR2 and SR2 is further coupled to SR3. In certain embodiments, H1 is directly coupled to SR1, SR1 is directly coupled to SR16, SR16 is directly coupled to SR17, SR17 is directly coupled to SR23, SR23 is directly coupled to SR24, and SR24 is directly coupled to H4.

In certain embodiments, the regulatory cassette is selected from the group consisting of a CK8 promoter and a cardiac troponin T (cTnT) promoter. In certain embodiments, the protein encoded by the micro-dystrophin gene has between five spectrin-like repeats and eight spectrin-like repeats. In certain embodiments, the protein encoded by the micro-dystrophin gene has at least 80% or 90% sequence identity to the amino acid sequence of SEQ ID NO: 4 or 5 in WO2016/115543A2 (incorporated herein by reference).

Diseases or conditions having a potential to benefit from the rAAV comprising the subject modified VPI capsids include: Huntington's disease, X-linked myotubular myopathy (XLMTM), Acid maltase deficiency (e.g., Pompe disease), Spinal Muscular Atrophy (SMA), Myasthenia Gravis (MG), Amyotrophic lateral sclerosis (ALS), Friedreich's ataxia, Mitochondrial myopathy, Muscular dystrophies (Duchenne's muscular dystrophy, Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), Mucopolysaccharidoses (MPS), Metachromatic leukodystrophy (MLD), Batten Disease, Rett Syndrome, Krabbe Disease, Canavan discase, X-Linked Retinoschisis, Achromatopsia (CNGB3 and CNGA3), X-Linked Retinitis Pigmentosa, Age-Related Macular Degeneration, neovascularized macular degeneration, Pompe, Fabry's disease, MPS I, II, IIIA, IIIB, Gaucher's disease, Dannon Disease, AlAt Deficiency, Friedreich ataxia, Wilson's Disease, Batten Disease (CLN1, CLN3, CLN6, CLN8), Wolman Disease, Tay-Sachs, Niemann-Lick Type C, CDKL5 deficiency Disorder, B-thalassemia, Sickle cell disease.

In certain embodiments, diseases or conditions having a potential to benefit from the rAAV of the invention may include: Becker muscular dystrophy (BMD), Congenital muscular dystrophies (CMD), Bethlem CMD, Fukuyama CMD, Muscle-eye-brain diseases (MEBs), Rigid spine syndromes, Ullrich CMD, Walker-Warburg syndromes (WWS), Duchenne muscular dystrophy (DMD), Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), Motor neuron diseases including ALS (amyotrophic lateral sclerosis), Spinal-bulbar muscular atrophy (SBMA), Spinal muscular atrophy (SMA).

In certain embodiments, diseases or conditions having a potential to benefit from the rAAV of the invention may include ion channel diseases, which are typically marked by muscular weakness, absent muscle tone, or episodic muscle paralysis. They include Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia.

In certain embodiments, diseases or conditions having a potential to benefit from the rAAV of the invention may include mitochondrial diseases, which occur when structures that produce energy for a cell malfunction. Such diseases include: Friedreich's ataxia (FA), Mitochondrial myopathies, Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO).

In certain embodiments, diseases or conditions having a potential to benefit from the rAAV of the invention may include myopathies, which is a disease of muscle in which the muscle fibers do not function properly, resulting in muscular weakness. Myopathies include: Cap myopathies, Centronuclear myopathies, Congenital myopathies with fiber type disproportion, Core myopathies, Central core disease, Multiminicore myopathies, Myosin storage myopathies, Myotubular myopathy, Nemaline myopathies, Distal myopathies, GNE myopathy/Nonaka myopathy/hereditary inclusion-body myopathy (HIBM), Laing distal myopathy, Markesberg-Griggs late-onset distal myopathy, Miyoshi myopathy, Udd myopathy/tibial muscular dystrophy, Vocal cord and pharyngeal distal myopathy, Welander distal myopathy, Endocrine myopathies, Hyperthyroid myopathy, Hypothyroid myopathy, Inflammatory myopathies, Dermatomyositis, Inclusion-body myositis, Polymyositis, Metabolic myopathies, Acid maltase deficiency (AMD, Pompe disease), Carnitine deficiency, Carnitine palmityl transferase deficiency, Debrancher enzyme deficiency (Cori disease, Forbes disease), Lactate dehydrogenase deficiency, Myoadenylate deaminase deficiency, Phosphofructokinase deficiency (Tarui disease), Phosphoglycerate kinase deficiency, Phosphoglycerate mutase deficiency, Phosphorylase deficiency (McArdle disease), Myofibrillar myopathies (MFM), Scapuloperoncal myopathy.

In certain embodiments, diseases or conditions having a potential to benefit from the rAAV of the invention may include neuromuscular junction diseases, which result from the destruction, malfunction or absence of one or more key proteins involved in the transmission of signals between muscles and nerves. Such diseases include: Congenital myasthenic syndromes (CMS), Lambert-Eaton myasthenic syndrome (LEMS), Myasthenia gravis (MG).

In certain embodiments, diseases or conditions having a potential to benefit from the rAAV of the invention may include peripheral nerve diseases, in which the motor and sensory nerves that connect the brain and spinal cord to the rest of the body are affected, causing impaired sensations, movement or other functions. Such diseases include: Charcot-Marie-Tooth disease (CMT), Giant axonal neuropathy (GAN), muscle wasting in cachexia and aging.

4. Production of rAAV

The rAAV having the modified VPI capsids of the invention can be produced by using any standard rAAV production methods, typically using a producer cell line, so long as the method/producer cell line is modified to provide the modified VPI capsid proteins in place of (or at least in addition to) the wild-type VPI capsids.

A number of strategies differing in principles have been used for rAAV production, all of which can be used to produce the subject rAAV.

In certain embodiments, the subject rAAV is produced based on the helper-virus-free transient transfection method, with all cis and trans components (vector plasmid and packaging plasmids, along with helper genes isolated from adenovirus) in suitable host cells such as 293 cells. The transient-transfection method is simple in vector plasmid construction and generates high-titer AAV vectors that are free of adenovirus. The modified VP1 capsid proteins can be encoded by one of the plasmids used in transient transfection of the producer cell line.

In certain embodiments, the subject rAAV is produced using a recombinant herpes simplex virus (rHSV)-based AAV production system, which utilizes rHSV vectors to bring the AAV vector and the Rep and Cap genes (i.e., the modified VPI capsid gene of the invention) into the producer cells. The modified cap gene can be present in the rHSV vector that may also hosts the rAAV genome.

In certain embodiments, the subject rAAV is produced using a baculovirus system that requires simultaneous infection of insect cells with several baculovirus vectors to deliver the AAV vector cassette and the Rep and Cap genes (i.e., the modified VPI capsid gene of the invention).

In certain embodiments, the subject rAAV is produced based on certain AAV producer cell lines derived from, e.g., HeLa or A549 or HEK293 cells, which stably harbored AAV Rep/cap genes (i.e., the modified VPI capsid gene of the invention). The AAV vector cassette can either be stably integrated in the host genome or be introduced by an adenovirus that contained the cassette.

In certain embodiments, such producer cell line for rAAV production comprises an rAAV provirus that encodes the GOI flanked by the AAV ITR sequences, wherein the rAAV provirus is integrated into the genome of the producer cell line for rAAV production.

5. Treatment of Muscular Dystrophy Using rAAV

The subject rAAV comprising the modified VPI capsids of the invention can be used in gene therapy for treating various forms of muscular dystrophy, such as Duchenne's muscular dystrophy (DMD), Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), etc. In certain embodiments, the muscular dystrophy is DMD or BMD.

Thus one aspect of the invention provides a method of treating muscular dystrophy (such as DMD and BMD) in a subject in need thereof, the method comprising administering to the subject a therapeutically effective amount of a recombinant AAV (rAAV) vector encoding a functional version of the gene defective in the muscular dystrophy, such as a microdystrophin gene, wherein the rAAV comprises any of the modified VPI capsid proteins of the invention.

In certain embodiments, the microdystrophin gene is one described in U.S. Pat. Nos. 7,906,111; 7,001,761; 7,510,867; 6,869,777; 8,501,920; 7,892,824; PCT/US2016/013733; or U.S. Pat. No. 10,166,272 (all incorporated herein by reference).

In certain embodiments, the microdystrophin gene comprises a coding sequence for the R1, R16, R17, R23, and R24 spectrin-like repeats of the full-length dystrophin protein (such as one described in PCT/US2016/013733).

In certain embodiments, the method further comprises producing the subject rAAV prior to administering to the subject the rAAV so produced.

### EXAMPLES

### Example 1 In Vitro Assays Comparing the Subject AAV-SLB101 to AAV9 and AAV8

This experiment compares the effect of the subject rAAV capsid on expression of a GOI from a recombinant AAV vector in a muscle cell, in comparison to that of AAV8 and AAV9.

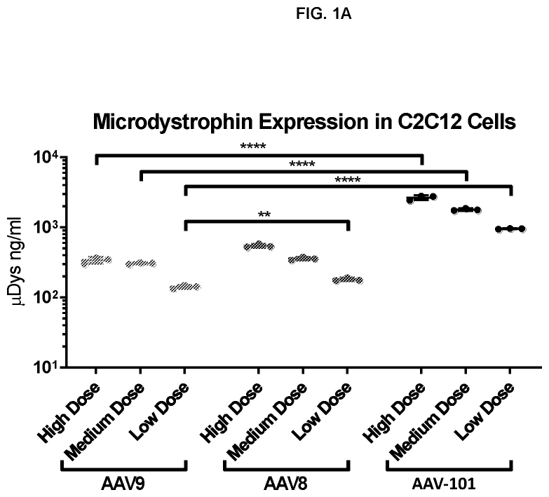

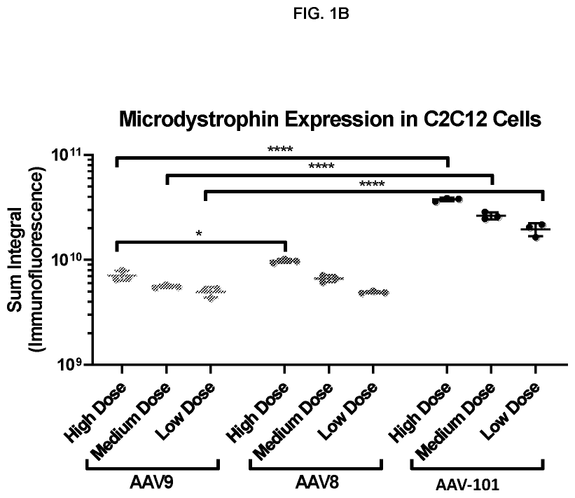

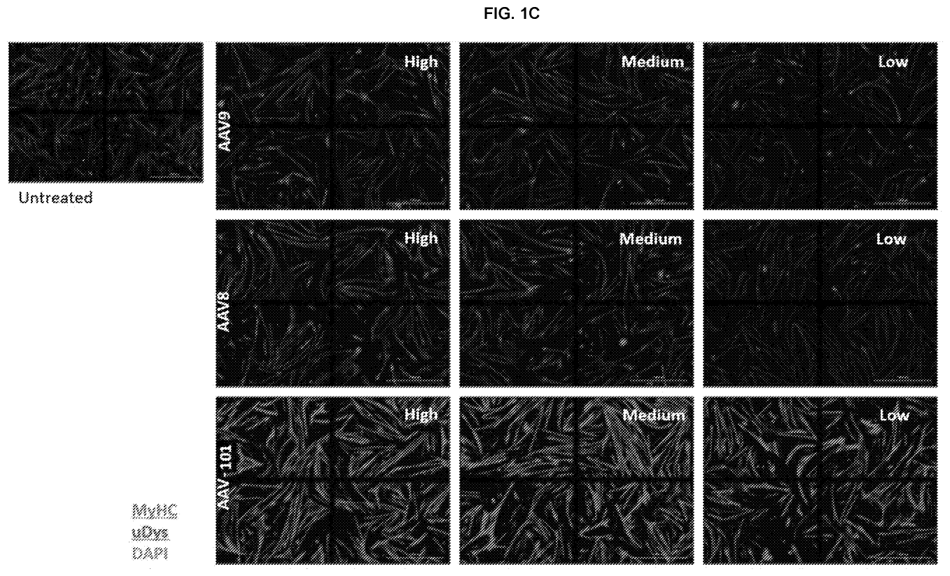

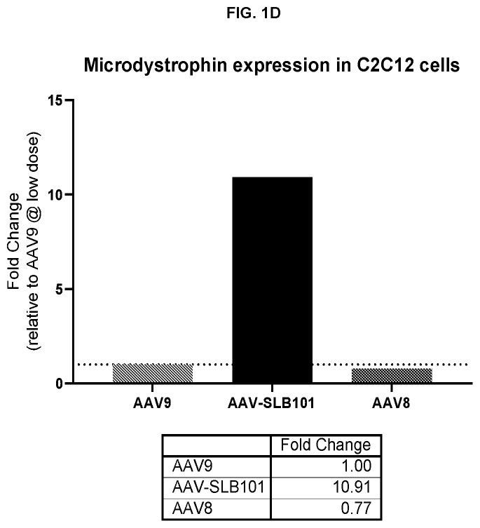

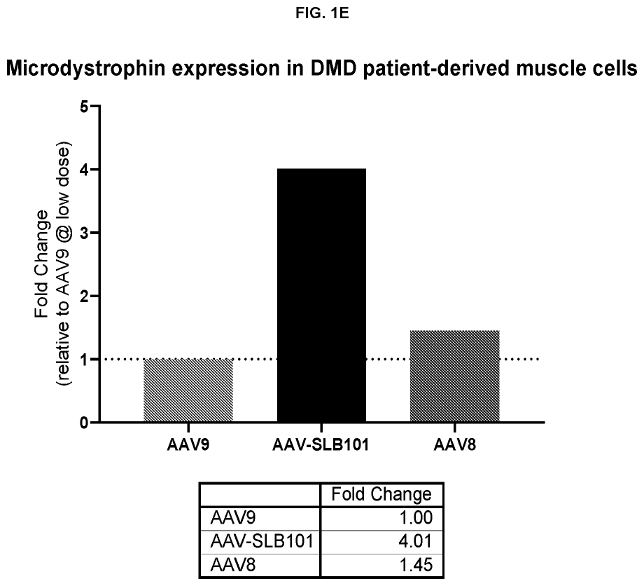

In particular, C2C12 cells—immortalized mouse myoblast cell line cells—were transduced at three multiplicities of infection (MOIs) with microdystrophin-expressing rAAV vectors packaged in AAV9, AAV8, and the subject capsid AAV-SLB101 (SEQ ID NO: 14), respectively. Cells were harvested 72 hours after transduction, and microdystrophin expression was measured by ELISA. The data were shown in FIGS. 1A-1C, with mean+SD for each data point in FIGS. 1A and 1B. Statistical significance was determined by one-way ANOVA test in comparison to AAV9 data at each MOI.

In FIG. 1A, microdystrophin expression was determined based on micrograms (μg) of expressed microdystrophin per mL. The data shows that the subject rAAV capsid was associated with much higher expression of the GOI (e.g., microdystrophin) in C2C12 muscle cells compared to AAV9 capsid. Although no direct statistical comparison has been made, the data also suggests that the subject rAAV capsid was also associated with much higher expression of the GOI (e.g., microdystrophin) in C2C12 muscle cells compared to AAV8 capsid.

In FIG. 1B, microdystrophin expression was determined based on sum integral of immunofluorescence intensity. The data again shows that the subject rAAV capsid was associated with much higher expression of the GOI (e.g., microdystrophin) in C2C12 muscle cells compared to AAV9 capsid. Although no direct statistical comparison has been made, the data also suggests that the subject rAAV capsid was also associated with much higher expression of the GOI (e.g., microdystrophin) in C2C12 muscle cells compared to AAV8 capsid.

In FIG. 1C, C2C12 cells were similarly transduced at three multiplicities of infection (MOIs) with microdystrophin-expressing rAAV vectors packaged in AAV9, AAV8, and the subject capsid AAV-SLB101, respectively. Cells were fixed and immunostained 72 hours after transduction, and microdystrophin expression was visualized by immunofluorescence (IF). Uninfected/untreated cells were stained with green fluorescence against Myosin heavy chain (MyHC). Cell nuclei were stained with DAPI. The data visually confirms the superior ability of the subject rAAV capsid to direct expression of the GOI (e.g., microdystrophin) in C2C12 muscle cells compared to both the AAV9 and AAV8 capsids, at all dose levels tested.

In another experiment, C2C12 cells were transduced with the same three AAVs above, but at the lowest MOI only. Cells were harvested 96 hours after transduction, and microdystrophin expression was measured. The data were then normalized to that of AAV9 and plotted in FIG. 1D, and fold change was indicated in the table insert in FIG. 1D. It is apparent that AAV-SLB101 has significantly higher microdystrophin protein expression than AAV9 (p<0.0001). Statistics were determined by ordinary one-way ANOVA.

In yet another experiment, patient-derived DMD cells were transduced with the same three AAVs above, but at the lowest MOI only. Cells were harvested 72 hours after transduction, and microdystrophin expression was measured. The data were then normalized to that of AAV9 and plotted in FIG. 1E, and fold change was indicated in the table insert in FIG. 1E. It is apparent that AAV-SLB101 has significantly higher microdystrophin protein expression than AAV9 (p<0.0001). Statistics were determined by ordinary one-way ANOVA.

### Example 2 In Vitro Potency Assay on C2C12 and DMD Cells

C2C12 cells were transduced at three doses of multiplicity of infection (MOI) with microdystrophin-expressing rAAV vectors packaged in AAV9, AAV8, and the subject capsid AAV-SLB101, respectively. Cells were harvested 72 hours after transduction, and microdystrophin expression was measured. The data shown are mean+SD. Statistical analysis was determined by one-way ANOVA in comparison to AAV9 at each MOI.

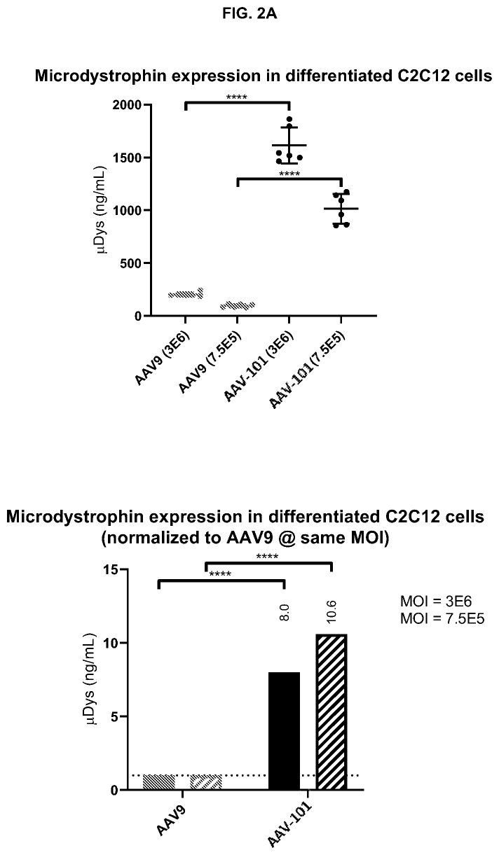

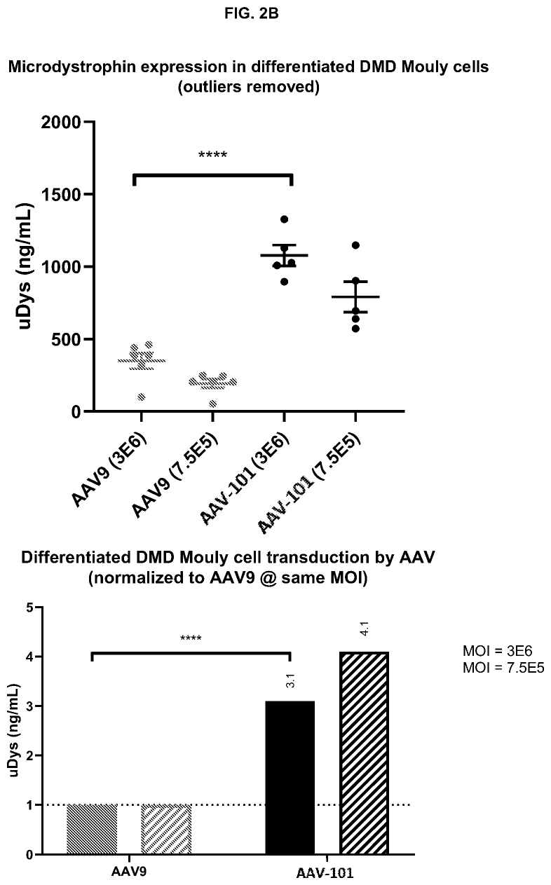

In FIG. 2A, the top panel shows statistically significantly more microdystrophin expression, in two doses (3E6 and 7.5E5), in differentiated C2C12 cells by using rAAV with the subject rAAV capsid, compared to rAAV with AAV9 capsid. In FIG. 2A, the bottom panel shows the same result in bar graph after normalizing to AAV9 at the same MOI. Specifically, at the higher dose of 3E6, expression level was 8.0-fold higher with the subject capsid. At the lower dose of 7.5E5, expression level was 10.6-fold higher with the subject capsid.

Similar experiments were repeated in patient-derived DMD cells—Mouly cells.

Specifically, patient-derived DMD Mouly cells were transduced at three multiplicities of infection (MOIs) with microdystrophin-expressing vectors packaged in AAV9, AAV8, and the subject capsid AAV-SLB101, respectively. Cells were harvested 72 hours after transduction, and microdystrophin expression was measured. The data shown are mean+SD. Statistical analysis was determined by one-way ANOVA in comparison to AAV9 at each MOI.

In FIG. 2B, the top panel shows statistically significantly more microdystrophin expression, in two doses (3E6 and 7.5E5), in differentiated DMD Mouly cells by using rAAV with the subject rAAV capsid, compared to rAAV with AAV9 capsid. Outliers were removed from the data. In FIG. 2B, the bottom panel shows the same result in bar graph after normalizing to AAV9 at the same MOI. Specifically, at the higher dose of 3E6, expression level was 3.1-fold higher with the subject capsid. At the lower dose of 7.5E5, expression level was 4.1-fold higher with the subject capsid.

### Example 3 In Vivo Comparison of the Subject AAV-SLB101 Capsid to AAV9

The mouse model of DMD—mdx mice— were used in this experiment to demonstrate the superior expression level of microdystrophin in rAAV having the subject capsid, compared to that with AAV9 capsid.

About 5-6 weeks old mdx mice were systemically injected (intravenous delivery) with microdystrophin-expressing rAAV vectors packaged in the subject capsid AAV-SLB101, or in AAV9 capsid, at a dose of 1E14 vg/kg. The mice were necropsied 2-4 weeks post injection (N=3 at 2 weeks, and N=4 at 4 weeks), and tissues were harvested for quantification of vector biodistribution and microdystrophin expression. Statistics in all panels were determined by individual Welch's t tests in comparison to AAV9.

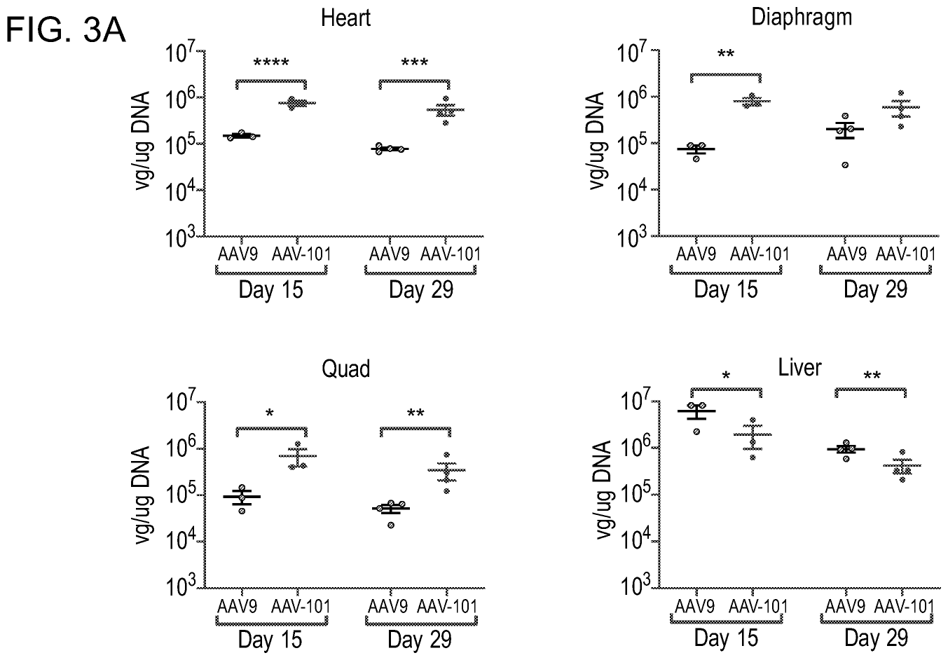

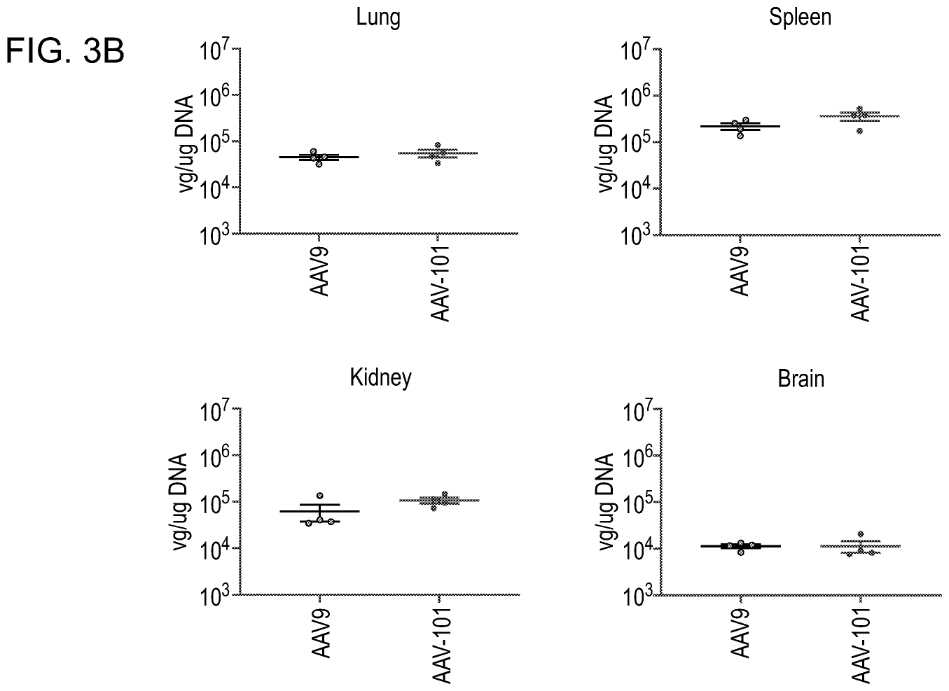

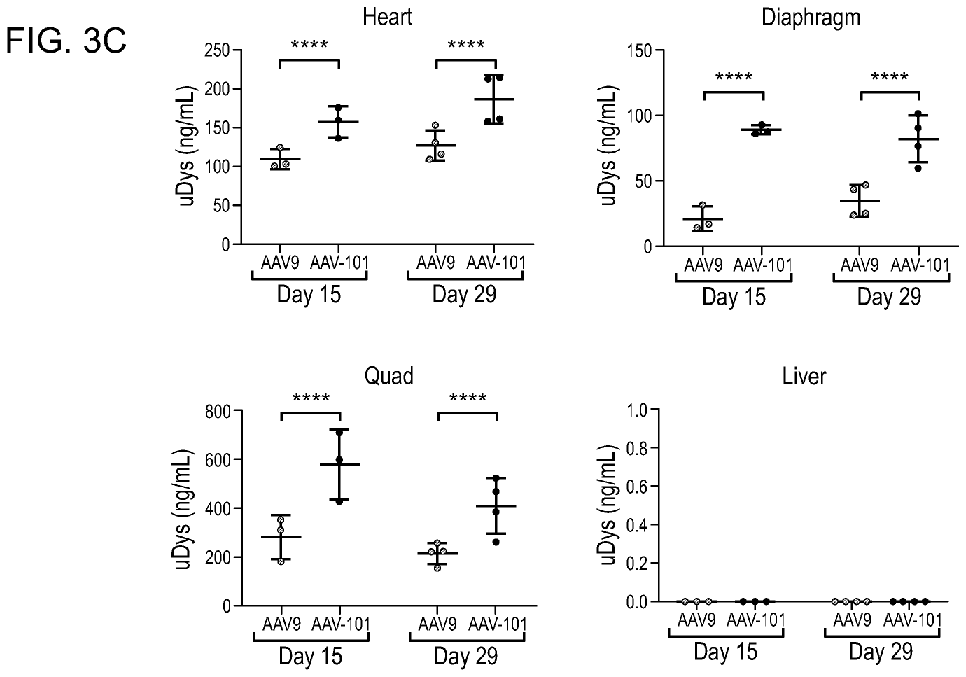

FIG. 3A shows that AAV-SLB101 has a significantly higher biodistribution in heart (p<0.001) and quadriceps (“quad”) (p<0.01) in one or both time points (i.e., Day 15 and Day 29), and is trending towards higher vg in diaphragm (particularly at Day 15). Meanwhile, AAV-SLB101 has significantly lower vg in liver than AAV9 (p<0.01). The data shown are mean+SD. Statistical analysis was determined by one-way ANOVA in comparison to AAV9 at each time point. No significant difference was found in the brain (data not shown).

FIG. 3B shows that there is no observable difference in expression in multiple peripheral tissues at Day 29, between rAAV in the subject capsid and rAAV in AAV9 capsid, as expected due to the use of a muscle-specific promoter. The tested peripheral tissues include lung, spleen, kidney, and brain.

FIG. 3C shows that microdystrophin expression is significantly higher with rAAV packaged in the subject AAV-SLB101 capsid than rAAV packaged in AAV9 capsid, in all three muscle tissues assayed (i.e., heart (cardiac muscle) (p<0.05), diaphragm (smooth muscle), and quad (skeletal muscle)) (p<0.05), at both Day 15 and Day 29. There is no discernible expression in liver for both constructs. The data shown are mean+SD. Statistical analysis was determined by one-way ANOVA in comparison to AAV9 at each time point.

The above data demonstrates that the subject AAV-SLB101 capsid has superior GOI (e.g., microdystrophin) expression efficiency, in both cultured C2C12 muscle cells, as well as patient-derived DMD Mouly cells, in in vitro potency assays. The in vitro results were also confirmed by in vivo biodistribution study conducted in the mouse DMD model mdx mice.

Specifically, in vivo biodistribution study in mdx mice shows that the subject AAV-SLB101 capsid has a significantly higher biodistribution over AAV9 in heart and quad in both time points tested (i.e., Day 15 and Day 29), and is trending towards higher vg in diaphragm particularly at Day 15. Further, the subject AAV-SLB101 capsid has significantly lower vg in liver, an unintended target tissue for microdystrophin expression.

In terms of microdystrophin expression, the subject AAV-SLB101 capsid has significantly higher level of expression then AAV9 in all three muscle tissues tested.

### Example 4 Characterization of Additional Modified AAV-SLB Capsids

Additional modified AAV9 capsids, including AAV-SLB102 to AAV-SLB112, as well as two modified capsides with similar insertion sequences by different surrounding/flanking sequences (AAV-SLB113 and AAV-SLB-114), were constructed and tested for their potency in comparison to wild-type AAV9 and AAV-SLB101. The insertion sequences of AAV-SLB102 to AAV-SLB112 are SEQ ID NOs: 2-12, respectively. See FIG. 6 for a multi-sequence alignments of the tested constructs in relation to wild-type AAV9 sequence and the AAV-SLB101 sequence.

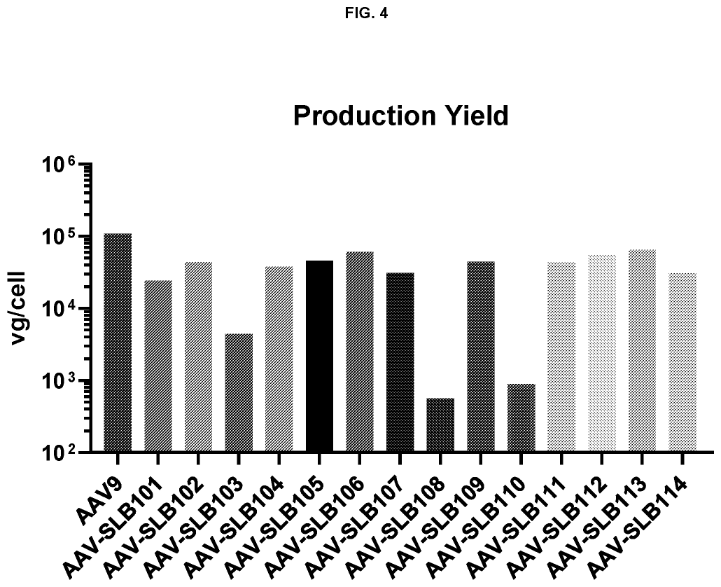

In the first experiment, production yield of the various capsid constructs were compared to that of wild-type AAV9 capsid as well as that of AAV-SLB101, based on triple transfection of adherent 293T cells, followed by purification via step-wise iodixanol ultracentrifugation. The measured production yields were plotted in FIG. 4. With a few exceptions, most constructs had similar (but slightly lower) yields compared to wild-type AAV9.

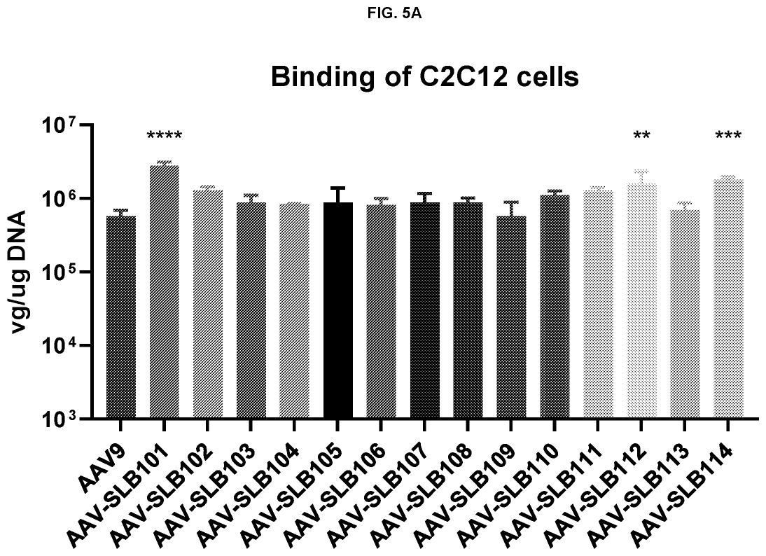

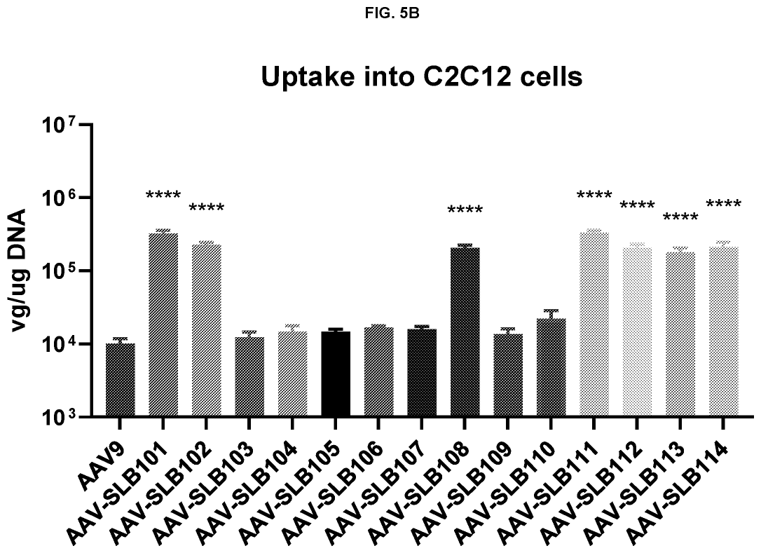

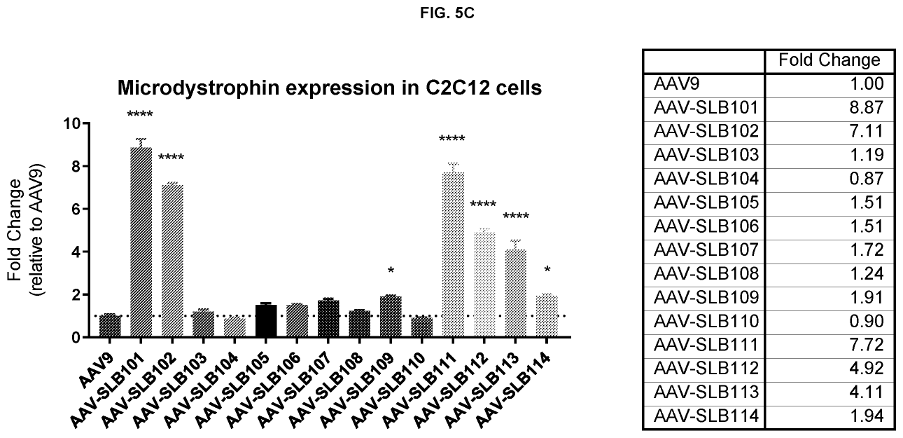

A series of in vitro characterization experiments were performed to compare the additional subject modified AAV9 capsids with wild-type AAV9 and the previously tested AAV-SLB101 in C2C12 cells. The results were reported in FIGS. 5A-5C.

Specifically, quantification of AAV capsid binding to the cell surface of C2C12 cells was measured by qPCR of DNA isolated after 1 hour of incubation at 4° C. The results in FIG. 5A shows that AAV-SLB101 (p<0.0001), 112 (p<0.01) and 114 (p<0.001) all bound to C2C12 cells significantly more than wild-type AAV9, while the remaining constructs all had similar binding compared to wild-type AAV9. Statistics are determined by ordinary one-way ANOVA.

However, there appeared to be a dramatic difference in uptake by C2C12 cells of the somewhat equally bound viral vectors with different modified capsids. Specifically, in FIG. 5B, quantification of uptake of AAV into C2C12 cells was measured by qPCR of DNA isolated after 1 hour of incubation at 4° C. followed by an additional hour at 37° C. The results showed that AAV-SLB101, 102, 108, 111, 112, 113, 114 were taken up by C2C12 cells significantly more (about 20-40 fold more) than AAV9 (p<0.0001). Other tested constructs appeared to have comparable cellular uptake compared to AAV9. Statistics are determined by ordinary one-way ANOVA.

Finally, in FIG. 5C, C2C12 cells were transduced with microdystrophin-expressing vectors packaged in AAV9, AAV-SLB101 and the thirteen additional modified AAV capsids. Cells were harvested 96 hours after transduction, and microdystrophin expression was measured. The data were normalized to that of AAV9, and fold change was indicated in the table insert in FIG. 5C. It is apparent that AAV-SLB101, 102, 111, 112 and 113 had the highest microdystrophin protein expression over AAV9 (p<0.0001), with AAV-SLB109 and 114 resulting in only slightly higher expression than AAV9 (p<0.001). Statistics are determined by ordinary one-way ANOVA.

Notably, despite the fact that SLB101, SLB113, and SLB114 all have the same insertion sequence RGDLGLS, and differ primarily in the flanking sequences, SLB101 appears to lead to much significantly higher microdystrophin-expressing level (8.87: 4.11: 1.94). That is, SLB101 is 116% more than that of SLB113, which itself is 112% more than that of SLB114.

In summary, the above data shows that at least one of the subject modified AAV9 capsides, AAV-SLB101, showed superior efficiency in comparison to wild-type AAV9 in in vitro assays in both mouse and DMD human skeletal muscle cells. This also translated to increased biodistribution and microdystrophin protein expression in vivo in both quadriceps & heart, and decreased biodistribution to liver, in comparison to AAV9. An expanded panel of the subject modified AAV9 capsids also identified at least two additional candidates interest, AAV-SLB102 and AAV-SLB111, that are similar to AAV-SLB 101 in in vitro assays for binding, uptake, and microdystrophin protein expression in C2C12 cells.

### Example 5 Functional Importance of the Sequences Surrounding the Inserted Peptides

This example demonstrates the surprising finding that the functions of the modified AAV9 capsids depend on not only the identity of the short peptides inserted into the wild-type AAV9 capsid protein, but also the surrounding sequences of the inserted short peptides. Functional comparison was made using three representative modified AAV9 capsids -SLB-101, SLB-113, and SLB-114. See sequence alignments in FIG. 6. All three sequences comprise the same 7-residue core sequence RGDLGLS, but differ slightly in the immediate N- and C-terminal sequences surrounding the 7-residue core sequence. In SLB-101, the 7-residue core sequence was inserted between residues 588 and 589 of the wild-type AAV9 VPI sequence (i.e., there is no change of any wild-type AAV9 VPI sequence other than the inserted 7-residue core sequence). See local sequence alignment between wild-type AAV9 and SLB-101 in FIG. 6.

In contrast, in SLB-113 and SLB-114, there are additional changes both within the immediately N-terminal 3-4 residues and within the immediately C-terminal 4-5 residues, with respect to the 7-residue core sequence (see FIG. 6). Overall, the sequences of SLB-113 and SLB-114 appear to be more similar to each other compared to that of SLB-101.

FIG. 5A shows that, in vitro, the ability of these capsids to bind C2C12 cells differs, with SLB-101 and SLB-114 both binding significantly more strongly to C2C12 cells compared to wild-type AAV9, yet SLB-113 showed no significant difference.

In FIG. 5B, C2C12 cells infected by AAV9 variants SLB-101 appeared to express the encoded microdystrophin at a surprisingly higher level than that in SLB-113 and SLB-114.

In order to show that such in vitro results were not due to artifacts associated with the cell line used (i.e., C2C12 cells), in vivo expression studies were designed using the model DMD^{mdx} mice. Specifically, 5-6 weeks old mdx mice were injected with a single dose of 1E14 vg/kg of either wild-type AAV9, or AAV9 with the SLB-101, -102, -113, or -114 variant capsids. SLB-102 was included, although it has a different inserted short peptide (Sec FI. 6). Injected mice were necropsied 2-4 weeks post injection (N=3 @ 2 weeks, and N=3-4 @ 4 weeks). Biodistribution of the virus and microdystrophin expression via MSD were examined in the four groups of mice compared to the wild-type control group.

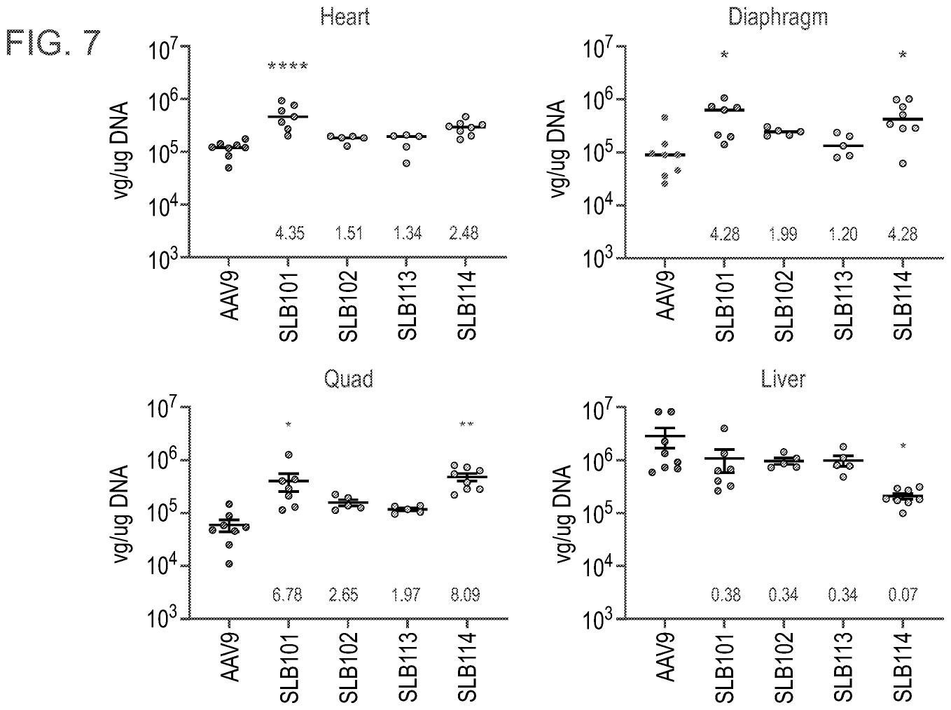

FIG. 7 shows that SLB-101 and SLB-114 both have significantly increased biodistribution to the quadriceps (skeletal) muscles compared to wild-type AAV9 (6.78-fold and 8.09-fold, respectively). Meanwhile, the modest increases in SLB-102 and -113 are statistically insignificant. Similar observation was also made in the diaphragm (skeletal muscle).

In the heart, SLB-101 biodistribution is significantly higher than that of wild-type AAV9 (4.35-fold). SLB-114 also trended high (2.48-fold) but was not statistically significant. Again, SLB-102 and -113 both had about the same distribution compared to that of the AAV9 control.

Interestingly, although all AAV9 variants had lower liver distribution, only SLB-114 was statistically significantly lower. See FIG. 7.

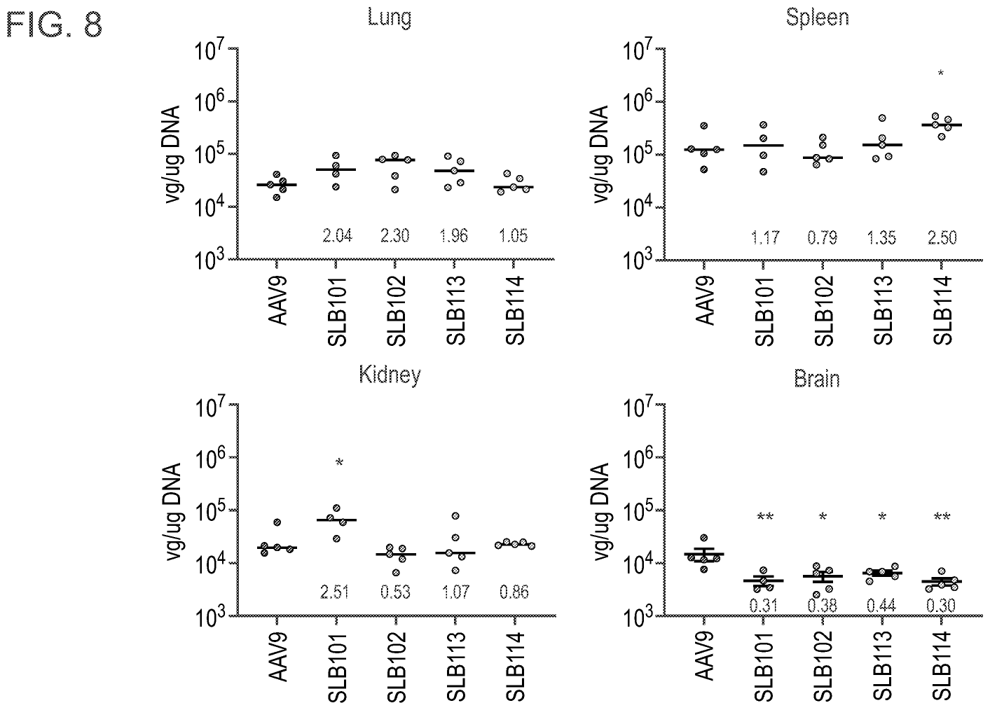

Extended biodistribution in a number of other organs was also examined. FIG. 8 shows that all had statistically significantly lower distribution in the brain, while distribution in the lung, spleen and kidney appeared to be comparable to that of wild-type AAV9, except that SLB-101 was higher in the kidney, and SLB-114 was higher in the spleen.

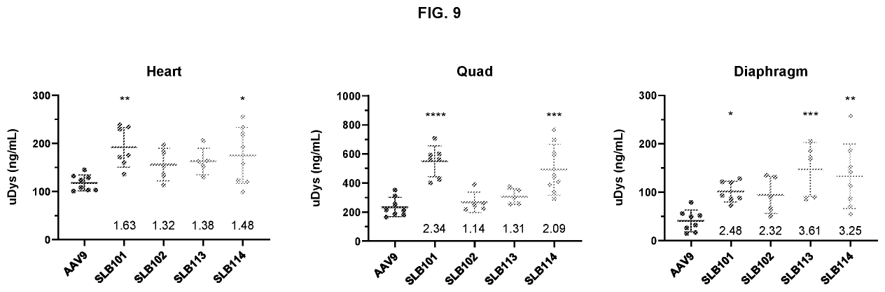

Microdystrophin expression was examined in the heart, the quad, and the diaphragm in FIG. 9. Both SLB-101 and SLB-114 had statistically higher expression than wild-type AAV9 in all three tissues, while SLB-102 was unremarkable everywhere. Meanwhile, SLB-113 was only higher in the diaphragm, but was not statistically significantly higher than AAV9 in the two other tissues.

These data showed that at least biodistribution and microdystrophin expression were affected by the surrounding sequences, since SLB-101, -113 and -114 all have the same 7-residue core sequence insertion.

1. A modified adeno-associate virus (mAAV) capsid polypeptide comprising a polypeptide selected from the group consisting of SEQ ID NOs: 1-12, wherein the polypeptide is inserted between residues 588 and 589 of wild-type AAV9 VP1 capsid, or the two corresponding residues of a non-AAV9 wild-type Clad F (e.g., AAVhu.32), a wild-type Clad A (e.g., AAV6), or a wild-type Clad E (e.g., AAV8, AAVrh.10, or AAVrh.74) VP1 capsid.

2. The mAAV capsid polypeptide of claim 1, comprising the polypeptide of SEQ ID NO: 1, 11, or 12.

3. A modified adeno-associate virus (mAAV) capsid polypeptide comprising, consisting essentially of, or consisting of the polypeptide of SEQ ID NO: 1 11 or 12.

4-5. (canceled)

6. A recombinant adeno-associated virus (rAAV), comprising the mAAV capsid polypeptide of claim 1.

7. The rAAV of claim 6, wherein:
the VP1 capsid of the rAAV consists of or consists essentially of the mAAV capsid polypeptide of claim 1; and/or
the rAAV comprises a gene of interest (GOI) flanked by a pair of AAV ITR sequences.

8. (canceled)

9. The rAAV of claim 7, wherein the pair of AAV ITR sequences are AAV2 or AAV9 ITR sequences.

10. The rAAV of claim 7, wherein the gene of interest (GOI) includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 21), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

11. (canceled)

12. The rAAV of claim 10, wherein the GOI encodes a microstrophin, wherein:
the microdystrophin is one described in U.S. Pat. Nos. 7,906,111; 7,001,761; 7,510,867; U.S. Pat. Nos. 6,869,777; 8,501,920; 7,892,824; PCT/US2016/013733; or U.S. Pat. No. 10,166,272;
the microdystrophin comprises a coding sequence for R16 and R17 spectrin-like repeats for the full length dystrophin protein (such as one described in U.S. Pat. No. 7,892,824); or
the microdystrophin comprises a coding sequence for the R1, R16, R17, R23, and R24 spectrin-like repeats of the full-length dystrophin protein; or a microdystrophin gene described in PCT/US2016/013733 or U.S. Pat. No. 10,479,821.

13-14. (canceled)

15. The rAAV of claim 7, wherein the GOI is operatively linked to a transcriptional regulatory cassette, such as a muscle specific promoter (e.g., a CK8 promoter or a cardiac troponin T (cTnT) promoter).

16. The rAAV of claim 7, wherein the GOI is a micro-dystrophin gene encoding a protein comprising, from N- to C-terminus, an amino-terminal actin-binding (AB1) domain, a β-dystroglycan binding domain, a Hinge 1 domain (H1), a spectrin-like repeat domain consisting of five spectrin-like repeats that include spectrin-like repeat 1 (SR1), spectrin-like repeat 16 (SR16), spectrin-like repeat 17 (SR17), spectrin-like repeat 23 (SR23), and spectrin-like repeat 24 (SR24), and a Hinge 4 domain (H4), wherein the micro-dystrophin gene is operatively linked to a muscle-specific human muscle creatine kinase CK8 promoter (e.g., SEQ ID NO:19 of U.S. Pat. No. 10,479,821), and wherein the GOI is flanked by a pair of AAV2 ITR (inverted terminal repeat) sequence.

17. A polynucleotide encoding the modified adeno-associate virus (mAAV) capsid polypeptide of claim 1, a polypeptide sequence at least 95%, 96%, 97%, 98%, or 99% identical thereto.

18. The polynucleotide of claim 17, which is codon-optimized for mammalian expression.

19. A vector comprising the polynucleotide of claim 17.

20. The vector of claim 19, which is a plasmid or a viral vector (such as an HSV vector, or an AAV vector).

21. A cultured host cell comprising:
a recombinant nucleic acid molecule encoding: (1) the modified adeno-associate virus (mAAV) capsid polypeptide of claim 1, or (2) a sequence at least 95%, 96%, 97%, 98%, or 99% identical to (1), wherein the recombinant nucleic acid molecule optionally further comprises a heterologous non-AAV sequence.

22. A method of treating a disease or condition such as muscular dystrophy in a subject in need thereof, the method comprises administering to the subject a therapeutically effective amount of the rAAV of claim 10.

23. The method of claim 22, wherein compared to an otherwise identical reference rAAV with wild-type AAV9 VPI capsid, the GOI of the rAAV is preferentially expressed in cardiac muscle, skeletal muscle, and/or smooth muscle (e.g., smooth muscle in diaphragm).

24. The method of claim 22, wherein compared to an otherwise identical reference rAAV with wild-type AAV9 VPI capsid, the GOI of the rAAV is expressed in liver at a statistically significantly lower level.

25-26. (canceled)

27. A method of producing rAAV, wherein the rAAV comprises the mAAV capsid polypeptide of claim 1, the method comprising introducing a rAAV vector encoding a GOI flanked by a pair of ITR sequences to a producing or packaging cell line expressing the mAAV capsid polypeptide of claim 1.

28. The method of claim 27, wherein the producing or packaging cell line is infected by an HSV vector encoding the mAAV capsid polypeptide of claim 1, or transfected with a sequence encoding the mAAV capsid polypeptide of claim 1.

29-30. (canceled)


SEQUENCE LISTING (38 sequences)

SEQ ID NO: 1 - protein, 7 aa, Artificial Sequence
  Description of Artificial Sequence Synthetic peptide
RGDLGLS

SEQ ID NO: 2 - protein, 7 aa, Artificial Sequence
  Description of Artificial Sequence Synthetic peptide
RGDMSRE

SEQ ID NO: 3 - protein, 7 aa, Artificial Sequence
  Description of Artificial Sequence Synthetic peptide
GEARISA

SEQ ID NO: 4 - protein, 7 aa, Artificial Sequence
  Description of Artificial Sequence Synthetic peptide
ESGLSQS

SEQ ID NO: 5 - protein, 7 aa, Artificial Sequence
  Description of Artificial Sequence Synthetic peptide
EYRDSSG

SEQ ID NO: 6 - protein, 7 aa, Artificial Sequence
  Description of Artificial Sequence Synthetic peptide
DLGSARA

SEQ ID NO: 7 - protein, 7 aa, Artificial Sequence
  Description of Artificial Sequence Synthetic peptide
SGNSGAA

SEQ ID NO: 8 - protein, 9 aa, Artificial Sequence
  Description of Artificial Sequence Synthetic peptide
CDCRGDCFC

SEQ ID NO: 9 - protein, 7 aa, Artificial Sequence
  Description of Artificial Sequence Synthetic peptide
NDVRSAN

SEQ ID NO: 10 - protein, 7 aa, Artificial Sequence
  Description of Artificial Sequence Synthetic peptide
NDVRAVS

SEQ ID NO: 11 - protein, 13 aa, Artificial Sequence
  Description of Artificial Sequence Synthetic peptide
GGGRGDLGLSGGG

SEQ ID NO: 12 - protein, 13 aa, Artificial Sequence
  Description of Artificial Sequence Synthetic peptide
GGSRGDLGLSGGS

SEQ ID NO: 13 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 743 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLGLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 749 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGGGRGDLGLSGGGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 749 aa, Artificial Sequence
  Description of Artificial Sequence Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGGSRGDLGLSGGSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 13 aa, Adeno-associated virus
NHQSAQAQAQTGW

SEQ ID NO: 18 - protein, 13 aa, Adeno-associated virus
NLQQQNTAPQIGT

SEQ ID NO: 19 - protein, 13 aa, Adeno-associated virus
NLQQQNAAPIVGA

SEQ ID NO: 20 - protein, 13 aa, Adeno-associated virus
NLQQQNTGPIVGN

SEQ ID NO: 21 - protein, 13 aa, Adeno-associated virus
NLQSSSTDPATGD

SEQ ID NO: 22 - protein, 49 aa, Artificial Sequence
  Description of Artificial Sequence Synthetic polypeptide
  MOD_RES (29)..(29): R, G, E, D, S, C, N, or absent
  MOD_RES (30)..(30): G, E, S, Y, L, D, or absent
  MOD_RES (31)..(31): D, A, G, R, N, C, V, S, or absent
  MOD_RES (32)..(32): L, M, R, D, S, or absent
  MOD_RES (33)..(33): G, S, I, A, or absent
  MOD_RES (34)..(34): L, R, S, Q, A, D, V, or absent
  MOD_RES (35)..(35): S, E, A, G, C, N, or absent
TNEEEIKTTNPVATESYGQVATNHQSAQXXXXXXXAQAQTGWVQNQGIL

SEQ ID NO: 23 - protein, 42 aa, Adeno-associated virus
TNEEEIKTTNPVATESYGQVATNHQSAQAQAQTGWVQNQGIL

SEQ ID NO: 24 - protein, 49 aa, Artificial Sequence
  Description of Artificial Sequence Synthetic polypeptide
TNEEEIKTTNPVATESYGQVATNHQSAQRGDLGLSAQAQTGWVQNQGIL

SEQ ID NO: 25 - protein, 49 aa, Artificial Sequence
  Description of Artificial Sequence Synthetic polypeptide
TNEEEIKTTNPVATESYGQVATNHQSAQRGDMSREAQAQTGWVQNQGIL

SEQ ID NO: 26 - protein, 49 aa, Artificial Sequence
  Description of Artificial Sequence Synthetic polypeptide
TNEEEIKTTNPVATESYGQVATNHQSAQGEARISAAQAQTGWVQNQGIL

SEQ ID NO: 27 - protein, 49 aa, Artificial Sequence
  Description of Artificial Sequence Synthetic polypeptide
TNEEEIKTTNPVATESYGQVATNHQSAQESGLSQSAQAQTGWVQNQGIL

SEQ ID NO: 28 - protein, 49 aa, Artificial Sequence
  Description of Artificial Sequence Synthetic polypeptide
TNEEEIKTTNPVATESYGQVATNHQSAQEYRDSSGAQAQTGWVQNQGIL

SEQ ID NO: 29 - protein, 49 aa, Artificial Sequence
  Description of Artificial Sequence Synthetic polypeptide
TNEEEIKTTNPVATESYGQVATNHQSAQDLGSARAAQAQTGWVQNQGIL

SEQ ID NO: 30 - protein, 49 aa, Artificial Sequence
  Description of Artificial Sequence Synthetic polypeptide
TNEEEIKTTNPVATESYGQVATNHQSAQSGNSGAAAQAQTGWVQNQGIL

SEQ ID NO: 31 - protein, 51 aa, Artificial Sequence
  Description of Artificial Sequence Synthetic polypeptide
TNEEEIKTTNPVATESYGQVATNHQSAQCDCRGDCFCAQAQTGWVQNQGIL

SEQ ID NO: 32 - protein, 49 aa, Artificial Sequence
  Description of Artificial Sequence Synthetic polypeptide
TNEEEIKTTNPVATESYGQVATNHQSAQNDVRSANAQAQTGWVQNQGIL

SEQ ID NO: 33 - protein, 49 aa, Artificial Sequence
  Description of Artificial Sequence Synthetic polypeptide
TNEEEIKTTNPVATESYGQVATNHQSAQNDVRAVSAQAQTGWVQNQGIL

SEQ ID NO: 34 - protein, 55 aa, Artificial Sequence
  Description of Artificial Sequence Synthetic polypeptide
TNEEEIKTTNPVATESYGQVATNHQSAQGGGRGDLGLSGGGAQAQTGWVQNQGIL

SEQ ID NO: 35 - protein, 55 aa, Artificial Sequence
  Description of Artificial Sequence Synthetic polypeptide
TNEEEIKTTNPVATESYGQVATNHQSAQGGSRGDLGLSGGSAQAQTGWVQNQGIL

SEQ ID NO: 36 - protein, 22 aa, Artificial Sequence
  Description of Artificial Sequence Synthetic polypeptide
NHQSGQAGRGDLGLSAQAATGW

SEQ ID NO: 37 - protein, 22 aa, Artificial Sequence
  Description of Artificial Sequence Synthetic peptide
NHQGQSGRGDLGLSAQAAQTGW

SEQ ID NO: 38 - protein, 20 aa, Artificial Sequence
  Description of Artificial Sequence Synthetic peptide
NHQSAQRGDLGLSAQAQTGW